(19) United States
(12) Patent Application Publication
(10) Pub. No.: US 2009/0177130 A1
(43) Pub. Date: Jul. 9, 2009

(54) DEEP PRESSURE METHODS, APPARATUS AND SYSTEMS FOR AUTISM THERAPY AND OTHER THERAPIES

(76) Inventor: Seth M. Wegher-Thompson, Thousand Oaks, CA (US)

Correspondence Address: HENRICKS SLAVIN AND HOLMES LLP;SUITE 200, 840 APOLLO STREET, EL SEGUNDO, CA, 90245, US

(21) Appl. No.: 11/952,279

(22) Filed: Dec. 7, 2007

(51) Int. Cl. A61H 7/00; A61B 17/00

(52) U.S. Cl. 601/151; 606/201

(57) ABSTRACT

Apparatus and methods for applying compressive force to portions of a person's body to, for example, provide therapy to an autistic person.

### BACKGROUND OF THE INVENTIONS

1. Field of Inventions

The present inventions relate generally to therapeutic methods, apparatus and systems that involve the application of deep pressure to, for example, treat autism.

2. Description of the Related Art

Autism is a developmental disability that typically appears during the first three years of life and is the result of a neurological disorder. Autism effects the normal functioning of the brain which, in turn, adversely impacts development in the areas of social interaction and communication. Individuals with autism also frequently have sensory difficulties, i.e. they may be hypo-reactive, hyper-reactive, or lack the ability to integrate the senses. Sensory integration therapy, which is usually done by occupational, physical or speech therapists, focuses on desensitizing the child and helping him or her reorganize sensory information. For example, if a child has difficulties with the sense of touch, sensory integration therapy might include handling a variety of materials with different textures.

Deep pressure therapy, which is sometimes referred to as “hug therapy” or “squeeze therapy,” is a sensory integration therapy that involves the application of pressure which is typically associated with firm touching, e.g. hugging, cuddling and squeezing. It has been found that deep pressure therapy is calming and reduces arousal in the nervous system. To that end, Temple Grandin, Ph.D. developed a “squeeze machine” (sometimes referred to a “hug machine”) that applies lateral, inwardly directed pressure to both lateral aspects of a person's entire body by compressing the user between two foam-padded panels.

The present inventor has determined that the conventional methods, apparatus and systems for applying deep pressure are susceptible to improvement. In particular, the present inventor has determined that conventional methods, apparatus and systems involve relatively large, expensive and immobile instrumentalities. The present inventor has also determined that it would be desirable to provide inward pressure to more than the lateral aspects of the person's body.

### BRIEF DESCRIPTION OF THE DRAWINGS

Detailed descriptions of exemplary embodiments of the inventions will be made with reference to the accompanying drawings.

FIG. 1 is a plan view of a therapeutic apparatus in accordance with one embodiment of a present invention in an uninflated state.

FIG. 2 is a perspective view of a portion of the therapeutic apparatus illustrated in FIG. 1.

FIG. 3 is a perspective view of the therapeutic apparatus illustrated in FIG. 1 in a partially inflated state.

FIG. 4 is a perspective view of the therapeutic apparatus illustrated in FIG. 1 in a fully inflated state.

FIG. 5 is a plan view of a therapeutic apparatus in accordance with one embodiment of a present invention in an uninflated state.

FIG. 6 is a plan view of a therapeutic apparatus in accordance with one embodiment of a present invention in an uninflated state.

### DETAILED DESCRIPTION OF THE EXEMPLARY EMBODIMENTS

The following is a detailed description of the best presently known modes of carrying out the inventions. This description is not to be taken in a limiting sense, but is made merely for the purpose of illustrating the general principles of the inventions. Additionally, although various embodiments of the present therapeutic apparatus, systems and methods are described below with reference to the treatment of autism, the present inventions also have application in other therapeutic areas. For example, the present therapeutic apparatus, systems and methods may be used to reduce anxiety, facilitate relaxation, or otherwise reduce a person's mental stress level, reduce lactic acid buildup in muscles, and to restrain a person (or animal) in various settings.

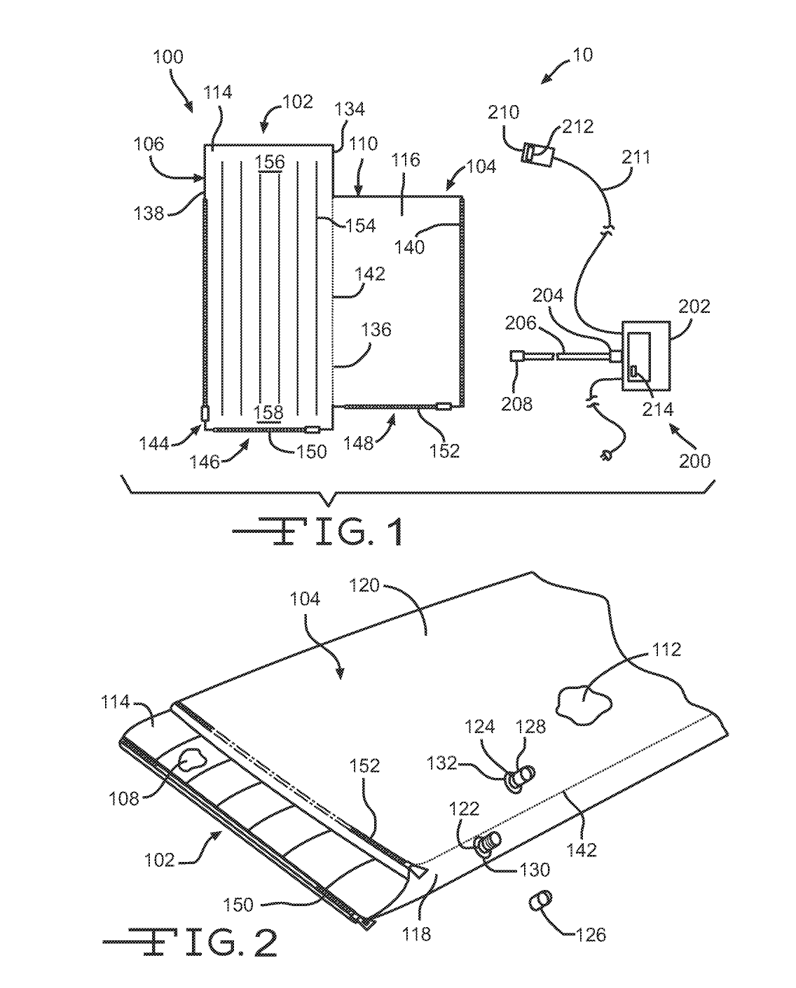

A therapeutic system in accordance with one embodiment of a present invention is generally represented by reference numeral 10 in FIG. 1 and consists of a therapeutic apparatus 100 and an inflation apparatus 200.

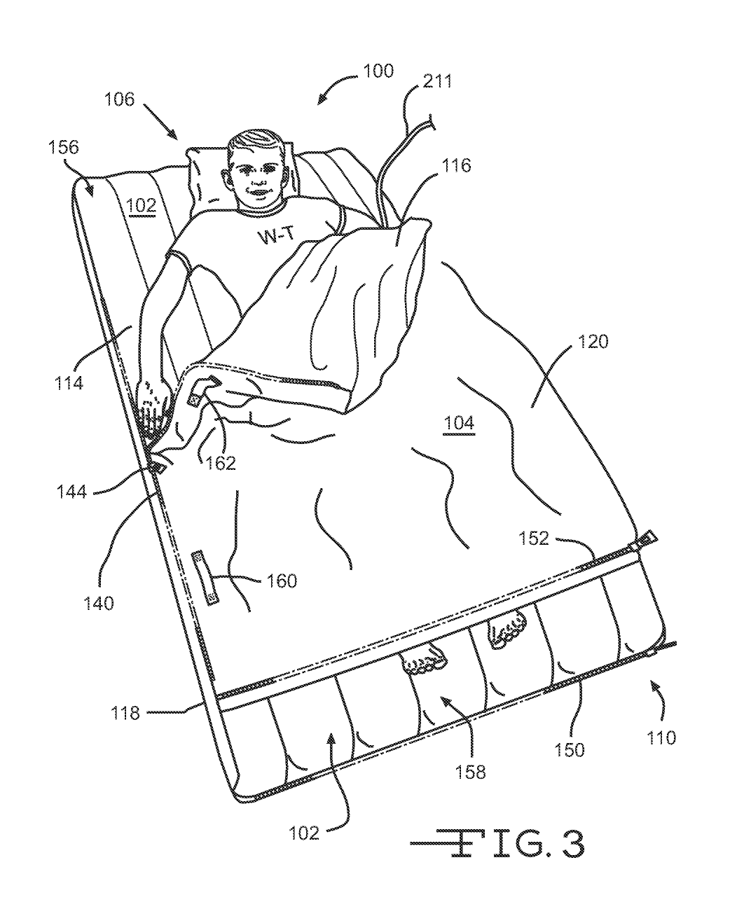

As illustrated in FIGS. 1 and 2, the exemplary therapeutic apparatus 100 includes an inflatable base 102 and an inflatable cover 104. The inflatable base 102 has an exterior shell 106 and an inner inflatable bladder 108 within the exterior shell, while the inflatable cover 104 has an exterior shell 110 and an inner inflatable bladder 112 within the exterior shell. In the illustrated embodiment, the exterior shells 106 and 110 are two-part structures that respectively include relatively soft, comfortable inner members 114 and 116 and durable outer members 118 and 120. Suitable materials for the inner members 114 and 116 include, but are not limited to, Lycra®, spandex and other elastic fabrics, satin woven fabrics formed from silk or polyester, felt, and cotton fabrics. Suitable materials for the outer members 118 and 120 include, but are not limited to, canvas fabric, nylon fabric, and other durable heavy duty fabrics. Turning to the inflatable bladders 108 and 112, there is no fluidic connection between the inflatable bladders in the illustrated embodiment and, to that end, the inflatable bladders 108 and 112 include connectors 122 and 124 with caps 126 and 128. The shell outer members 118 and 120 are provided with apertures 130 and 132 with which the connectors 122 and 124 are aligned and through which the connectors extend. Suitable materials for the inflatable inner bladders 108 and 110 include, but are not limited to, rubbers, plastics, and textile-reinforced plastics.

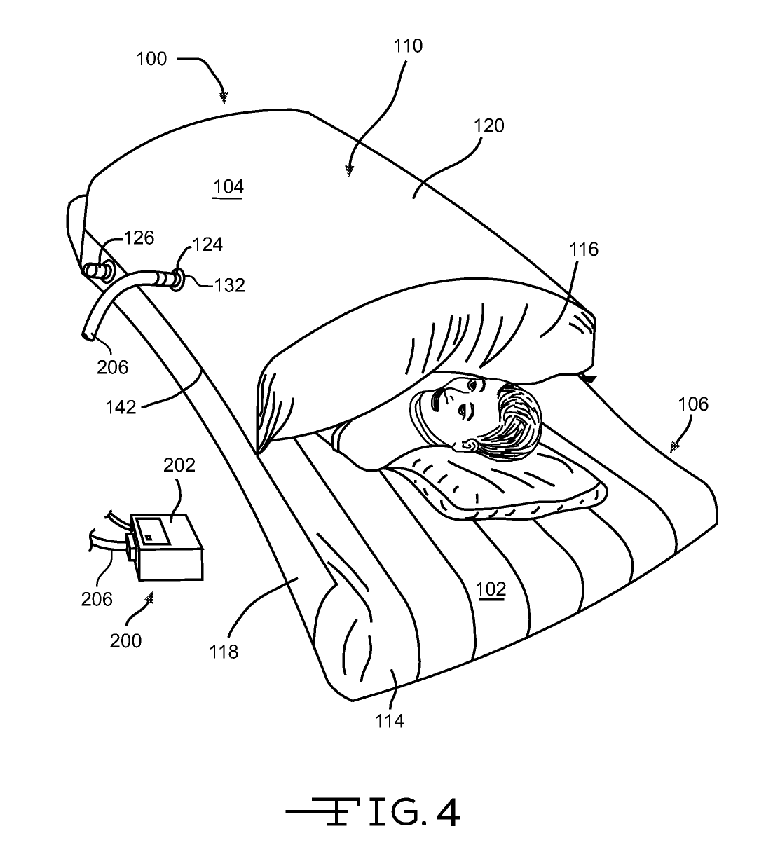

The base 102 and cover 104 of the exemplary therapeutic apparatus 100 illustrated in FIGS. 1 and 2 are connected to one another along their longitudinally extending side edges 134/136 and 138/140 during use, as is explained in greater detail below in the context of FIGS. 3 and 4. The longitudinally extending side edges 134 and 136 are secured to one another by a hinge (or joint) 142 that is formed when the base and cover exterior shells 106 and 110 are secured to one another (e.g. by stitching) during the assembly process. Alternatively, in those instances where the shell outer members 118 and 120 are together formed from a single piece of material, the hinge will be defined by the connections of the inner members 114 and 116 to the outer members 118 and 120 at the longitudinally extending side edges 134 and 136. In either case, the inflatable base 102 and inflatable cover 104 are permanently connected to one another at the longitudinally extending side edges 134 and 136. Another alternative is to provide a releasable connection, through the use of a fastener mechanism such as a zipper or hook and loop fastener strips (commonly sold under the trade name Velcro), to connect the longitudinally extending side edges 134 and 136 to one another. Turning to the longitudinally extending side edges 138 and 140, the edges 138 and 140 in the illustrated embodiment may be releasably connected to one another through the use of a fastener mechanism 144, such as the illustrated zipper or hook and loop fastener strips. Alternatively, the longitudinally extending side edges 138 and 140 may be permanently connected to one another along the entire length of the cover 104. Another alternative is to permanently connect the longitudinally extending side edges 138 and 140 to one another along a portion (e.g. half) of the length of the cover 104 and releasably connect the remainder with a fastener mechanism such as a zipper or hook and loop fastener strips.

Although the present inventions are not so limited, the inflatable inner bladders 108 and 112 in the exemplary embodiment illustrated in FIGS. 1 and 2 are removable and the exterior shells 106 and 110 are provided with openings 146 and 148 that have fastener mechanisms 150 and 152, e.g. the zippers (as shown), hook and loop fastener strips, buttons, snaps or the like, that secure the edges of the openings to one another. In other embodiments, the inflatable inner bladders 108 and 112 may be non-removable and the openings 146 and 148 may be omitted. Here, the exterior shells 106 and 110 may be respectively secured to the inflatable inner bladders 108 and 112. In still other embodiments, the exterior shells 106 and 110 may be omitted and the inflatable bladders 108 and 112 may be secured to one another in, for example, the same manner as the exterior shells and may also be configured to be relatively comfortable and durable.

In yet other therapeutic apparatus embodiments, a single inflatable bladder may be used in place of the individual inflatable bladders 108 and 112. For example, a single inflatable bladder may be placed within a single exterior shell that has a comfortable inner member and durable outer member to define a unitary base and cover. Such an exterior shell could have a fastener mechanism (e.g. fastener mechanism 144) associated with the longitudinally extending side edges to secure the therapeutic apparatus around the user. Alternatively, the exterior shell may be omitted and one longitudinally extending side edge of the single bladder, which itself defines a unitary base and cover, may be secured to the other in the same manner as the exterior shell or some other manner.

With respect to the configuration of the inflatable inner bladders 108 and 112, the inner bladder 108 of the base 102 consists of a single unit that is generally rectangular when viewed in plan and includes a plurality of pleats 154. In other implementations, the inner bladder 108 may consist of a plurality of individual units that are connected to one other, either directly or through the use of a suitable manifold, or which are not connected and each have their own connector 122. The pleats 154 may also be omitted if desired. The inner bladder 112 of the cover 104 also consists of a single unit that is generally rectangular in shape when viewed in plan. When viewed in a section taken perpendicular to the hinge 142, the inner bladder 112 is somewhat elliptical in shape in that the center bulges toward and away from the base 102 (note FIG. 4). Here too, a plurality of individual units, which are connected to one another directly or through the use of a suitable manifold, may be used in place of a single unit.

The respective lengths and relative positions of the inflatable base 102 and inflatable cover 104 result in the exemplary therapeutic apparatus 100 illustrated in FIGS. 1 and 2 defining head region 156, where the head will typically be located during use, and a foot region 158, where the feet will typically be located during use. Although the overall size of the inflatable base 102 may vary according to the size of the intended user, e.g. about 6 feet to about 7.5 feet in length and about 3 feet to about 4.5 feet in width for adults, and about 4.5 feet to about 6 feet in length and about 3 feet to 4.5 feet in width for children, the head region 156 will be about 1 foot to two feet in length and the foot region will be about 6 inches to about 1.5 feet in length in order to provide supports for the user's head and feet during use (note FIGS. 3 and 4). In other implementations, the head region 156 and/or foot region 158 may be omitted and the cover 104 will extend to the head end and/or foot end of the inflatable base 102. Here, if desired, the foot ends of the inflatable base 102 and inflatable cover 104 may permanently or releasably connected to one another in, for example, the manner described above with respect to the longitudinal side edges 138/140 of the opening 146.

Handles 160 and 162 (FIG. 3) may also be provided on the inflatable base 102 and/or inflatable cover 104.

Turning to the inflation apparatus 200, the inflation apparatus in the exemplary system 10 includes an air pump 202 with a hose connector 204, a hose 206 with a bladder connector 208 that is configured to releasably mate with the bladder connectors 122 and 124, and a handheld control 210 that is connected to the air pump by a cable 211. In the illustrated embodiment, the handheld control 210 has a three position switch 212 that is biased to the “off” position, where no inflation or deflation occurs, and can be moved to “inflate” and “deflate” positions by the user to selectively control the level of inflation and pressure applied. The air pump 202 also has an power on-off switch 214 and may also have other controls (not shown). Suitable air pumps include, but are not limited to, those commonly used to inflate and deflate inflatable mattresses. The inflation apparatus 200 may be powered by a wall outlet, batteries or other power source.

The exemplary inflation apparatus 200 may also be provided with various safety mechanisms. For example, the air pump 202 may be provided with a slow leak valve that allows air to escape from the inner bladder 108 and/or inner bladder 112 when the air pump is not inflating or deflating the inner bladders. Such a leak would allow the inner bladders to deflate, and the user to exit the therapeutic apparatus 100, in the event of a power failure, an air pump failure, or other emergency.

In other implementations, the inflation apparatus 200 may be provided with a pair of hose connectors 204 and hoses 206 in order to facilitate both separate and simultaneous inflation and deflation of the inner bladders 108 and 112. The exemplary inflations apparatus 200 may, in some implementations, also be permanently secured to the therapeutic apparatus 100 in a manner similar to some inflatable mattresses.

In addition to the therapeutic benefits described below, the present therapeutic system 10 is relatively inexpensive, lightweight and easily transportable. For example, due to the flexibility of the materials used to form the inflatable base 102 and the inflatable cover 104, the therapeutic apparatus 100 may be folded or rolled up for storage or transportation when deflated.

The exemplary therapeutic system 10 illustrated in FIGS. 1 and 2 may be employed in the manner illustrated in FIGS. 3 and 4 to provide deep pressure therapy to an autistic person. Depending on the order in which the following steps are performed, some or all of the steps may be performed by the autistic person or by a parent, therapist or other assistant (collectively “assistant”). Referring first to FIG. 3, the person may be positioned (or may position himself or herself) on the inflatable base 102 of the therapeutic apparatus 100 after the inner bladder 108 of the base has been inflated with, for example, the inflation apparatus 200. Alternatively, the base inner bladder 108 may be inflated after the person is on the inflatable base 102. The longitudinal edges 138 and 140 of the inflatable base 102 and inflatable cover 104 may be partially connected to one another by the fastener mechanism 144 (as shown), or completely connected, prior to the initial positioning so that the person can slide into the therapeutic apparatus 100. Alternatively, the longitudinal edges 138 and 140 of the inflatable base 102 and inflatable cover 104 may initially be completely disconnected and then connected to one another after the person is in position. The handheld control 208 may be held by the person in the therapeutic apparatus 100, so that he or she can control the inflation of the bladder 112 under the inflatable cover 104 (as shown), or may be held by the assistant. The inflation apparatus 200 may be connected to the connector 124 that is associated with the inner bladder 112 within the cover 104 (FIG. 4) at any time after the inflation of the inner bladder 108 within the base 102 has been inflated.

After the person is positioned within the therapeutic apparatus 100 in, for example, the manner illustrated in FIG. 4, the inflation apparatus 200 may be used to inflate the inner bladder 112 within the cover 104 to the desired pressure. Such inflation results in a full body squeeze, i.e. inward compression forces that extend 360 degrees around the head-to-toe axis of the body (i.e. around the longitudinally extending periphery of the body) from one end of the cover 104 to the other, and a full body deep pressure squeeze when sufficiently inflated. The person's arms may be located between the inflatable base 102 and inflatable cover 104 (as shown) or one or both of the person's arms may be outside the cover.

The method described above is, of course, susceptible to a wide degree of variation. By way of example, but not limitation, the bladder 112 within the cover 104 may be inflated prior to, or simultaneously with, the bladder 108 within the base 106.

As noted above, the present therapeutic system 10 may be employed in the manner described in the context of FIGS. 3 and 4 in areas other than the treatment of autism. For example, the present therapeutic system 10 may be used to reduce anxiety, facilitate relaxation, or otherwise reduce a person's mental stress level, reduce lactic acid buildup in muscles, and to restrain a person (or animal) in various settings. In each instance, the user will be placed in the therapeutic apparatus 100 in the manner described above.

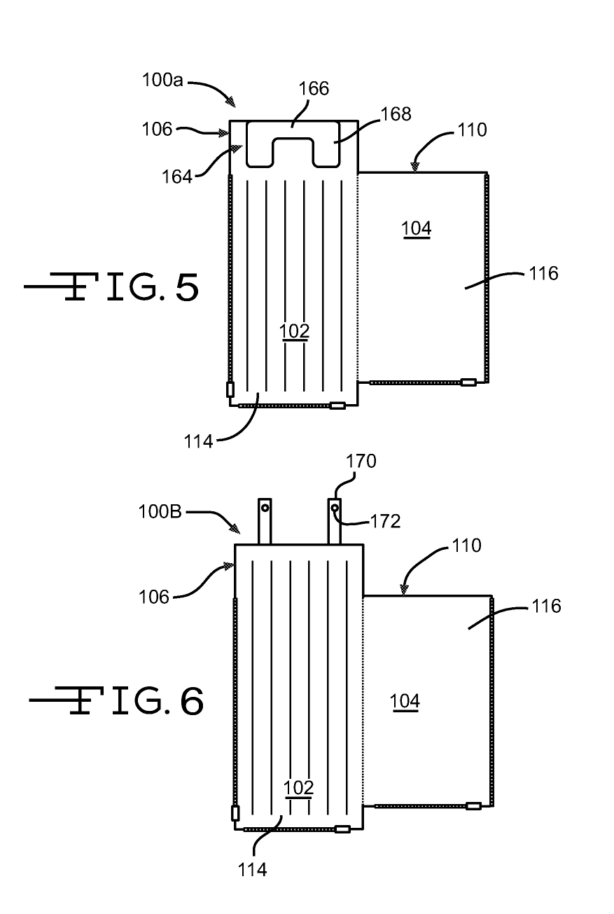

The exemplary therapeutic apparatus 100 may be reconfigured in a variety of ways. For example, the therapeutic apparatus 100a illustrated in FIG. 5 is substantially identical to therapeutic apparatus 100 and similar elements are represented by similar reference numerals. Here, however, the therapeutic apparatus 100a is provided with an inflatable hood 164 that may be used to provide compression to the head. The inflatable hood 164 is generally u-shaped, in that it includes a top portion 166 and side portions 168, and extends outwardly from the inflatable base 102 about 6 inches to about 1 foot. In the illustrated embodiment, the inflatable hood 164 is connected to the base inner bladder 108 and will inflate with the inner bladder. In other embodiments, the inflatable hood 164 may be a separate unit with its own inflation/deflation connector. The inflatable hood 164 may also be provided with a portion that extends over the forehead.

The therapeutic apparatus 100b illustrated in FIG. 6 is substantially identical to therapeutic apparatus 100 and similar elements are represented by similar reference numerals. Here, however, the therapeutic apparatus 100b is configured to also be employed in a vertical orientation with the user standing. To that end, a plurality of straps 170 which are configured to be secured to a wall or other support structure are attached to the inflatable base 102. Although other apparatus for securing the straps 170 to a wall may be employed, the exemplary straps have apertures 172 and may be hung on wall mounted hooks.

A number of additional features may also be added to the inflatable base 102 and/or inflatable cover 104 of the therapeutic apparatus 100-100b. For example, vibration elements or massage rollers may be added to the inflatable base 102 and/or cover 104. Heating and cooling mechanisms may be added to the inflatable base 102 and/or cover 104. Audio speakers and suitable audio connector(s) may be added to the inflatable base 102 and/or cover 104. With respect to the inflation apparatus 200, functionality such as the ability to preset the level of inflation for one or more users may be added.

Although the inventions disclosed herein have been described in terms of the preferred embodiments above, numerous modifications and/or additions to the above-described preferred embodiments would be readily apparent to one skilled in the art. By way of example, but not limitation, the inventions include any combination of the elements and/or step from the various species and embodiments disclosed in the specification that are not already described. By way of example, but not limitation, the bladders in the illustrated embodiment are inflated with air. In other embodiments, water or other liquids may be employed. In still other embodiments, an air/liquid combination (e.g. an air filled base and a water filled cover) may be employed. Here, a liquid pump may be used in place of, or in combination with, the air pump described above. It is intended that the scope of the present inventions extend to all such modifications and/or additions and that the scope of the present inventions is limited solely by the claims set forth below.

I claim:

1. An apparatus, comprising:
an inflatable base defining a size and a shape; and
an inflatable cover defining a size and a shape operably connected to the inflatable base;
the respective sizes and shapes of the inflatable base and the inflatable cover, and the operable connection thereof, being such that the inflatable base and the inflatable cover will apply inward pressure to at least a portion of a person's torso and at least a portion of the person's legs when inflated.

2. An apparatus as claimed in claim 1, wherein
the inflatable base and the inflatable cover each define first and second longitudinally extending side edges;
the first longitudinally extending side edges are permanently connected to one another; and
the second longitudinally extending side edges are releasably connected to one another.

3. An apparatus as claimed in claim 2, wherein
the second longitudinally extending side edge of the inflatable cover is releasably connected to the second longitudinally extending side edge of the inflatable base along the entire length of the second longitudinally extending side edge of the inflatable cover.

4. An apparatus as claimed in claim 1, wherein
at least one of the inflatable base and the inflatable cover includes an outer shell and a bladder within the outer shell.

5. An apparatus as claimed in claim 4, wherein
the outer shell includes a relatively soft inner member and a durable outer member.

6. An apparatus as claimed in claim 4, wherein
the bladder is removable.

7. An apparatus as claimed in claim 1, wherein
the inflatable base includes an outer shell and a bladder within the outer shell;
the inflatable cover includes an outer shell and a bladder within the outer shell; and
the base bladder and cover bladder are not connected to one another.

8. An apparatus as claimed in claim 1, wherein
the inflatable base defines a length;
the inflatable cover defines a length; and
the inflatable base is longer than the inflatable cover.

9. An apparatus as claimed in claim 8, wherein
the inflatable base defines a head end and a foot end;
the inflatable cover defines a head end and a foot end; and
the head end of the inflatable cover is longitudinally spaced from the head end of the inflatable base.

10. An apparatus as claimed in claim 9, wherein
the foot end of the inflatable cover is longitudinally spaced from the foot end of the inflatable base.

11. An apparatus as claimed in claim 1, wherein
the foot end of the inflatable cover and the foot end of the inflatable base together defining an opening.

12. An apparatus as claimed in claim 1, further comprising:
an inflatable hood associated with the inflatable base.

13. An apparatus as claimed in claim 1, further comprising:
at least one strap associated with the inflatable base and configured to secure the inflatable base to a support structure.

14. An apparatus as claimed in claim 1, further comprising:
a source of pressurized fluid.

15. A method of providing therapy to an autistic person, comprising the steps of:
positioning the autistic person within an inflatable structure; and
applying compressive force around the longitudinally extending periphery of at least a portion of the autistic person by inflating at least a portion of the inflatable structure.

16. A method as claimed in claim 15, wherein
the step of positioning the autistic person comprises positioning the autistic person within an inflatable structure in a prone position.

17. A method as claimed in claim 15, wherein
the step of positioning the autistic person comprises positioning the autistic person within an inflatable structure in a standing position.

18. A method as claimed in claim 15, wherein
the inflatable structure includes an inflatable base and an inflatable cover; and
the step of positioning the autistic person comprises positioning the autistic person within an inflatable structure after the inflatable base has been inflated.

19. A method as claimed in claim 18, wherein
the step of applying a compressive force comprises applying compressive force around the longitudinally extending periphery of at least a portion of the autistic person by inflating the inflatable cover after the autistic person has been positioned within the inflatable structure.

20. A method as claimed in claim 15, wherein
the step of applying a compressive force comprises applying compressive force around the longitudinally extending periphery of at least a portion of the autistic person's torso and at least a portion of the autistic person's legs by inflating at least a portion of the inflatable structure.

21. An apparatus, comprising:
an exterior shell; and
means, located within the exterior shell, for applying a compressive force around the longitudinally extending periphery of at least a portion of a person's torso and at least a portion of the person's legs in response to receipt of pressurized fluid.

22. An apparatus as claimed in claim 21, wherein the exterior shell comprises a relatively soft inner portion and a durable outer portion.

23. An apparatus as claimed in claim 21, wherein the pressurized fluid comprises pressurized gas.

24. A method of providing therapy to a person who desires to reduce his/her mental stress level, comprising the steps of:
positioning the person who desires to reduce his/her mental stress level within an inflatable structure; and
applying compressive force around the longitudinally extending periphery of at least a portion of the person who desires to reduce his/her mental stress level by inflating at least a portion of the inflatable structure.

25. A method as claimed in claim 24, wherein
the step of positioning comprises positioning the person who desires to reduce his/her mental stress level within an inflatable structure in a prone position.

26. A method as claimed in claim 24, wherein
the step of positioning comprises positioning the person who desires to reduce his/her mental stress level within an inflatable structure in a standing position.

27. A method as claimed in claim 24, wherein
the inflatable structure includes an inflatable base and an inflatable cover; and
the step of positioning comprises positioning the person who desires to reduce his/her mental stress level within an inflatable structure after the inflatable base has been inflated.

28. A method as claimed in claim 27, wherein
the step of applying a compressive force comprises applying compressive force around the longitudinally extending periphery of at least a portion of the person who desires to reduce his/her mental stress level by inflating the inflatable cover after the person has been positioned within the inflatable structure.

29. A method as claimed in claim 24, wherein
the step of applying a compressive force comprises applying compressive force around the longitudinally extending periphery of at least a portion of the torso of the person who desires to reduce his/her mental stress level and at least a portion of the person's legs by inflating at least a portion of the inflatable structure.
